(19) United States
(12) Patent Application Publication
(10) Pub. No.: US 2025/0205136 A1
(43) Pub. Date: Jun. 26, 2025

(54) HAIR SPRAY COMPOSITION COMPRISING CELLULOSE BLENDS AND PROCESS FOR PREPARING THE SAME

(71) Applicant: ISP Investments LLC, Wilmington, DE (US)

(72) Inventors: David C. STREULI, Wayne, NJ (US); Anthony Luschen, Wayne, NJ (US)

(73) Assignee: ISP Investments LLC, Wilmington, DE (US)

(21) Appl. No.: 18/849,420

(22) PCT Filed: Mar. 21, 2023
(86) PCT No.: PCT/US2023/015802

§ 371 Date: Sep. 20, 2024

Related U.S. Application Data
(60) Provisional application No. 63/322,081, filed on Mar. 21, 2022.

(51) Int. Cl. A61K 8/73; A61K 8/04; A61Q 5/06

(52) U.S. Cl. CPC A61K 8/731; A61K 8/046; A61Q 5/06

(57) ABSTRACT

Disclosed herein is an aqueous hair spray composition, the composition comprising: (i) 0.1 to 7.5 wt. % of a polymer blend comprising at least one nature-derived polymer selected from ethyl cellulose, hydroxypropyl cellulose and blends thereof; (ii) 0.1 to 99 wt. % of at least one solvent; and (iii) optionally 0.01 to 50 wt. % of at least one hair care additive. Further disclosed herein is an aerosol hair spray composition and a non-aerosol hair spray compositions comprising these polymer blends.

### FIELD OF THE INVENTION

The present disclosure relates to a hair spray composition comprising blends of ethyl cellulose, hydroxypropyl cellulose, synthetic polymer, alone or combinations thereof, and to a process for preparing the same.

### BACKGROUND OF THE INVENTION

Hair sprays traditionally have been used at the end of the styling process to fix the hair style in place and protect it from the elements such as humidity and wind. As the styling market and consumer behavior evolves, hair sprays are used as both finishing and working sprays. Finishing sprays are designed to be used as the last step in the styling process, spraying it on hair to hold the hair style in place, and protect it in humid/windy conditions, essentially freezing the hair style in place. These sprays are quick drying, and make hair less flexible, restricting the natural movement of hair. Often these hair sprays coat the hair with polymers that give a stiff, tacky feel to the hair. Working sprays are designed to be used as the hair style is created, and as such, they offer greater manageability, brushability and flexibility during styling. Working sprays have more water, with longer drying times to allow consumers to customize, texturize and create the hair style they desire. Ideal hair sprays (i) have good hold, (ii) show good retention of hair curls, (iii) be easily removed upon shampooing, (iv) be less sticky in nature and (v) and do not have visible residue or flaking on the hair fiber. Hair sprays typically come in aerosol form, but there are non-aerosol pump sprays as well. The challenge with polymers used in aerosol hair sprays is that the polymers need to be compatible and stable in presence of propellants and be soluble in alcohol and water. Synthetic polymers are widely used for their outstanding hold and humidity resistance, as well as propellant and solvent compatibility in aerosol formulations. While providing the desired benefits, these polymers do not fit the sustainability criteria put forward by governmental and no-governmental entities. As such, there is increasing pressure on manufacturers of hair sprays to provide formulations which meet the expected benefits, while using nature-derived, sustainable ingredients. As consumers are looking for environmentally friendly and sustainable products, the hair spray segment is struggling to offer nature-derived, sustainable alternatives to synthetic polymers. Many nature-derived polymers with the desired sustainability and biodegradability profiles are difficult to incorporate into hair spray formulations, due to restriction on their natural solubility and compatibility with solvents and propellants.

U.S. Pat. No. 10,722,447 assigned to ISP Investments LLC discloses sprayable composition comprising complex of (A) at least one charged (or pseudo-charged) polymer having a molecular weight of about 125,000 amu or more; and (B) a least one oppositely charged, rheology modifying, crosslinked polymer having at least one carboxylic functional group. The composition may provide a spray with a median particle size of less than about 175 um.

US Patent Application No.'s US2004-0180067 & US2004-0180068, both assigned to International Flavors & Fragrances discloses a particle composition containing a continuous phase of 0.5-95% organically soluble cellulosic material selected from the group consisting of ethyl cellulose and hydroxypropyl cellulose dissolved in 5-99.5% organic fragrance chemicals and organic flavor chemicals. The particles are formed from a continuous phase product that results from dissolving the cellulosic material in the flavor/fragrance which is subsequently subjected to techniques to form the desired particle size.

In this application, present inventors attempt to prepare nature-derived polysaccharide based polymers that can provide hold and humidity resistance to hair when blended or added to traditional synthetic polymers.

### SUMMARY OF THE INVENTION

The primary aspect of the present application is to provide a hair spray composition comprising: (i) 0.1 to 7.5 wt. % of a polymer blend comprising at least one nature-derived polymer selected from ethyl cellulose, hydroxypropyl cellulose and blends thereof; (ii) 0.1 to 99 wt. % of at least one solvent; and (iii) optionally 0.01 to 50 wt. % of at least one hair care additive.

Accordingly, the hair spray composition optionally further comprises 0.1 to 7.5 wt. % of at least one synthetic polymer selected from the group consisting of vinyl pyrrolidone/vinyl caprolactam/dimethylaminoethyl methacrylate copolymer, vinyl acetate/mono n-butyl maleate/isobornyl acrylate copolymer, N-tert-octylacrylamide, methylmethacrylate/acrylic acid/hydroxypropyl methacrylate/butyl aminoethyl methacrylate polymer, isobutylene/imidized maleic anhydride with ethylamine and ethanolamine, vinyl pyrrolidone/vinyl caprolactam/dimethylaminopropyl acrylamide copolymer, alkylated pyrrolidone (butyl), methyl vinyl ether/half ester of maleic anhydride (ethyl or butyl), vinyl pyrrolidone (PVP)/vinyl acetate/vinyl pyrrolidone copolymer, hydroxypropyl cellulose, hydroxethyl cellulose, octylacrylamide/acrylates/butyl aminoethyl methacrylate copolymer, acrylates/octylacrylamide copolymer, acrylates copolymer, Polyurethane-14 and AMP-acrylates copolymer, acrylates/hydroxypropyl epoxidized soybean oil copolymer, vinyl acetate/crotonates/vinyl neodecanoate copolymer, acrylates/octylacrylamide copolymer, t-butyl acrylate/ethyl acrylate/methacrylic acid, t-butyl acrylate/methacrylic acid/Dimethicone Copolyol, vinyl acetate/crotonic acid/vinyl neodecanoate, vinyl acetate/crotonic acid copolymer, copolymer of isophthalic acid, adipic acid, hexylene glycol, neopentyl glycol, dimethylol propanoic acid, and isophorone diisocyanate monomers, Polyacrylate-22, polyvinyl caprolactam, acrylates/t-butylacrylamide copolymer, acrylates/hydroxyesters acrylates copolymer, acrylates/C1-2 succinates/hydroxy acrylates copolymer, styrene/acrylates copolymer, pyrrolidone carboxylic acid salt of Chitosan, acrylates copolymer, methacryloyl ethyl betaine/acrylates copolymer, methacryloyl ethyl betaine/acrylates copolymer, and Polyester-5.

Another aspect of the present application is to provide a method for styling hair, the method comprising applying to human hair, 0.10 to 10 wt. % of a hair spray composition (i) 0.1 to 7.5 wt. % of a polymer blend comprising at least one nature-derived polymer selected from ethyl cellulose, hydroxypropyl cellulose and blends thereof; (ii) 0.1 to 99 wt. % of at least one solvent; and (iii) optionally 0.01 to 50 wt. % of at least one hair care additive.

Yet another aspect of the present application is to provide an aerosol hair spray composition comprising: (i) 0.1 to 7.5 wt. % of a polymer blend comprising at least one nature-derived polymer selected from ethyl cellulose, hydroxypropyl cellulose and blends thereof; (ii) 0.01 to 50 wt. % of at least one hair care additive; and (iii) 0.1 to 99.9 wt. % of at least one solvent; and (b) at least one propellant.

Yet another aspect of the present application is to provide a non-aerosol hair spray composition comprising: (i) 0.1 to 7.5 wt. % of a polymer blend comprising at least one nature-derived polymer selected from ethyl cellulose, hydroxypropyl cellulose and blends thereof; (ii) 0.01 to 50 wt. % of at least one hair care additive; and (iii) 0.1 to 99.9 wt. % of at least one solvent.

### BRIEF DESCRIPTION OF DRAWINGS

In addition to the cited advantages and objects of the disclosure, one or more descriptions of the disclosure briefly summarized can be added by reference to certain embodiments thereof which are illustrated in the appended drawings. These drawings form part of the specification. However, it is to be noted that the appended drawings illustrate preferred embodiments of the disclosure and therefore are not limiting in their scope.

FIG. 1 illustrates on hair performance and humidity resistance.

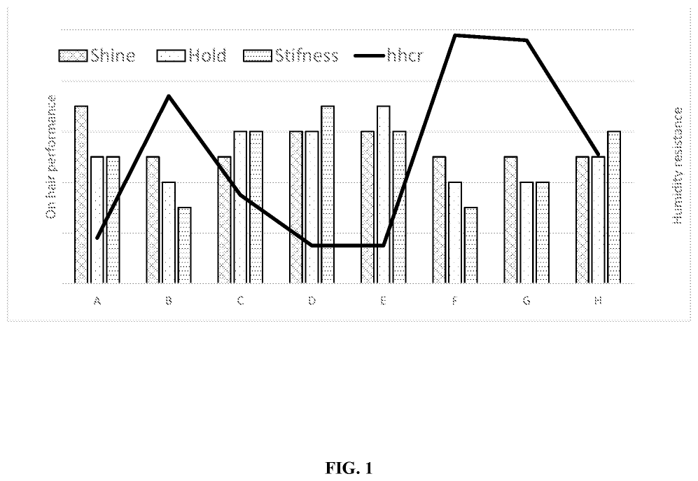

FIG. 2 illustrates Humidity Resistance of the HPC/EC Blend with a Synthetic Polymer at 80 F and 90% RH.

FIG. 3 illustrates on Hair Performance of the HPC/EC blend with a Synthetic Polymer.

### DETAILED DESCRIPTION OF THE INVENTION

The technical solutions of the present disclosure will be described clearly and better understood in combination with specific embodiments below, but those skilled in the art will understand that embodiments described below are part of embodiments of the present disclosure but not all of them and are only used for illustration of the present disclosure and should not be considered as limiting the scope of the disclosure. If any specific condition or process are not indicated in the examples, it is to be understood that the conditions are conventionally used by the manufacturer and is commercially available.

As utilized in accordance with the disclosure, the following terms, unless otherwise indicated, shall be understood to have the following meanings.

Unless otherwise defined herein, technical terms used in connection with the disclosed and/or claimed inventive concept(s) shall have the meanings that are commonly understood by those of ordinary skill in the art. Further, unless otherwise required by the context, singular terms shall include pluralities and plural terms shall include the singular.

The singular forms “a,” “an,” and “the” include plural forms unless the context clearly dictates otherwise specified or clearly implied to the contrary by the context in which the reference is made. The term “Comprising” and “Comprises of” includes the more restrictive claims such as “Consisting essentially of” and “Consisting of”.

For purposes of the following detailed description, other than in any operating examples, or where otherwise indicated, numbers that express, for example, quantities of ingredients used in the specification and claims are to be understood as being modified in all instances by the term “about”. The numerical parameters set forth in the specification and attached claims are approximations that may vary depending upon the desired properties to be obtained in carrying out the invention.

All percentages, parts, proportions, and ratios as used herein, are by weight of the total composition, unless otherwise specified. All such weights as they pertain to listed ingredients are based on the active level and, therefore; do not include solvents or by-products that may be included in commercially available materials, unless otherwise specified.

All publications, articles, papers, patents, patent publications, and other references cited herein are hereby incorporated herein in their entirety for all purposes to the extent consistent with the disclosure herein.

As used herein, the term “at least one” will be understood to include one as well as any quantity more than one, including but not limited to, 1, 2, 3, 4, 5, 10, 15, 20, 30, 40, 50, 100, etc. The term “at least one” may extend up to 100 or 1000 or more depending on the term to which it is attached. In addition, the quantities of 100/1000 are not to be considered limiting as lower or higher limits may also produce satisfactory results.

As used herein, the terms “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

As used herein, the term “Hydroxypropyl cellulose” or “HPC” refers to both commercially available products under the tradenames “Klucel E CS”, “Klucel L CS”, “Klucel G CS”, Ashland Inc., and in-house manufactured products.

As used herein, the term “cosmetically acceptable” refers to molecular entities regarded as safe, approved by regulatory body, listed in pharmacopoeia for use in topical contact with tissues (e.g., the skin) without undue toxicity, incompatibility, instability, irritation, allergic response, or the like. This term is not intended to limit the composition it describes as for use solely as a cosmetic (e.g., the composition may be used as a pharmaceutical).

As used herein, the term “polymer” refers to a compound comprising repeating structural units (monomers) connected by covalent chemical bonds. Polymers may be further derivatized, crosslinked, grafted or end-capped. Non-limiting examples of polymers include copolymers, terpolymers, tetra polymers, quaternary polymers, and homologues. The term “copolymer” refers to a polymer consisting essentially of two or more different types of monomers polymerized to obtain said copolymer.

As used herein, the term “personal care composition” and “cosmetics” refer to compositions intended for use on or in human body such as skin, hair, oral including those to alter the color and appearance of skin and hair.

As used herein, the term “nature-derived” refers to polymers prepared from natural sources like plants, bacteria, fungi or chemically modified natural polymers, wherein the building block monomer substance(s) and other substance(s) in the form of monomeric units and chemically bound substance(s) similarly originating from the natural polymers that has taken place in nature, independently of the extraction process with which they have been extracted. This means that “nature-derived polymers” are not necessarily ‘substances which occur in nature’.

In one non-limiting embodiment, the present disclosure is directed to a hair spray composition comprising: (i) 0.1 to 7.5 wt. % of a polymer blend comprising at least one nature-derived polymer selected from ethyl cellulose, hydroxypropyl cellulose and blends thereof; (ii) 0.1 to 99 wt. % of at least one solvent; and (iii) optionally 0.01 to 50 wt. % of at least one hair care additive.

Ethyl Cellulose is a linear polysaccharide derived from cellulose. Ethyl cellulose is similar in structure to cellulose but some of the hydroxyl (—OH) functional groups are replaced on the cellulose by the ethoxy group (—O-CH2-CH3) having the following structure:

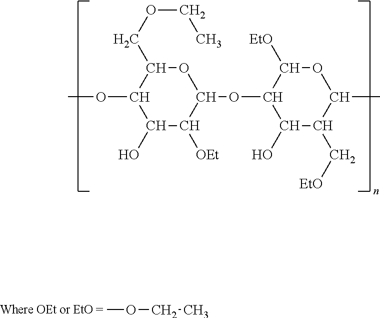

Ethyl cellulose finds wide applications in pharmaceuticals, cosmetics, vitamin coatings, printing inks, specialty coatings and food packaging. Commercially available ethyl cellulose polymers useful in embodiments of the invention include certain grades of AQUALON™ Ethyl Cellulose, available from Ashland Inc. Other commercially available forms of ethyl cellulose polymer which may be used in the invention include, for example, those available under the name ETHOCEL™, from The Dow Chemical Company. Various grades of Ethyl cellulose are available in the market being differentiated by physical characteristics such as molecular weight and degree of substitution.

Hydroxypropyl cellulose is a non-ionic cellulose ether with a versatile combination of properties. It is soluble in water at low temperatures and can combine drug solubility in aqueous and polar organic solvents, thermoplasticity, and surface activity with the thickening and stabilizing properties of other water-soluble cellulose polymers. It has the following structure:

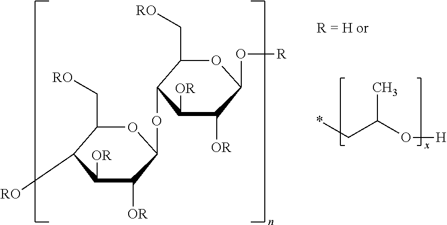

Commercially available hydroxypropyl cellulose useful in embodiments of the invention include certain grades of Klucel™ available from Ashland Inc. Other commercially available forms of hydroxypropyl cellulose polymer which may be used in the invention include, for example, those available under the name Benecel™ K100M, Nisso HPC SL, and the like.

In another non-limiting embodiment, the present hair spray composition further comprises optionally 0.1 to 5.0 wt. % of at least one synthetic polymer. Non-limiting examples of synthetic polymers used in hair spray composition is selected from the group consisting of Vinyl pyrrolidone/Vinyl Caprolactam/Dimethylaminoethyl methacrylate copolymer (Advantage LC, GaffixVC 713), vinyl acetate/mono n-butyl maleate/isobornyl acrylate copolymer (Advantage Plus), N-tert-Octylacrylamide, Methylmethacrylate/Acrylic acid/Hydroxypropyl methacrylate/Butyl amino ethyl methacrylate polymer (Advantage 4910), Isobutylene/imidized maleic anhydride with ethylamine and ethanolamine (Aquaflex FX-64), Vinyl pyrrolidone/Vinyl Caprolactam/Dimethylaminopropyl acrylamide copolymer (Aquaflex SF-40), Alkylated pyrrolidone (butyl) (Ganex), Methyl vinyl ether/half ester of maleic anhydride (ethyl or butyl) (Gantrez; Omnirez), Vinyl pyrrolidone (PVP)/Vinyl acetate/Vinyl pyrrolidone copolymer, modified cellulose (hydroxypropyl) (Klucel), modified cellulose (hydroxethyl) (Natrosol), Octylacrylamide/Acrylates/Butyl aminoethyl methacrylate copolymer (Amphomer 4910, Amphomer LV71, Balance 47), Acrylates/Octylacrylamide copolymer (Amphomer HC), Acrylates Copolymer (Balance CR), Polyurethane-14 and AMP-acrylates copolymer (DynamX), Acrylates/Hydroxypropyl Epoxidized Soybean Oil Copolymer (EXP hairspray), VA/Crotonates/Vinyl Neodecanoate Copolymer (Resyn28-2930), Acrylates/Octylacrylamide Copolymer (Resyn XP), t-butyl acrylate/ethyl acrylate/methacrylic acid (Luvimer 100P, 36D, 30E, pro 55), t-butyl acrylate/methacrylic acid/Dimethicone Copolyol (Luviflex Silk), Vinyl acetate/crotonic acid/Vinyl Neodecanoate (Luviset Can), Vinyl acetate/Crotonic acid copolymer (Luviset CA), copolymer of isophthalic acid, adipic acid, hexylene glycol, neopentyl glycol, dimethylol propanoic acid, and isophorone diisocyanate monomers (Luviset PUR), Polyacrylate-22 (Luviset Shape), Polyvinyl caprolactam (Luviskol Plus), acrylates/t-butylacrylamide copolymer (Ultrahold 8, Strong), acrylates/hydroxyesters acrylates copolymer (Acudyne 1000, DHR, 180), acrylates/C1-2 succinates/hydroxy acrylates copolymer (Acudyne LT-120), styrene/acrylates copolymer (Acudyne Shine), pyrrolidone carboxylic acid salt of Chitosan (Kytamer PC polymer), acrylates copolymer (Tilamar Fix A140, A1000), methacryloyl ethyl betaine/acrylates copolymer (Yukaformer), methacryolyl ethyl betaine/acrylates copolymer (Mihapol PBS-50), and polyester-5 (Aq. 38S, 48). Different cationic polymers such as polyquaternium compounds are also used. Polyquaternium is the International Nomenclature for Cosmetic Ingredients designation for several polycationic polymers that are used in the personal care industry. Different polymers are distinguished by the numerical value that follows the word “polyquaternium”. Polyquaternium-5, polyquaternium-7, and polyquaternium-47 are three examples, each a chemically different type of polymer.

Applicants have found after numerous experimentations, that combining ethyl cellulose with hydroxypropyl cellulose or a synthetic polymer brings about unique properties to the hair spray formulations. Hence the present application provides ethyl cellulose and hydroxypropyl cellulose polymer blend which is nature-derived and present in an amount of 0.1 to 5.0 wt. % of the total hair spray composition.

In another non-limiting embodiment, the present application provides polymer blends in different combinations. Accordingly, the polymer can be (i) a blend of ethyl cellulose polymers selected from the group consisting of (a) ethyl cellulose polymer having weight average molecular weight ranging from 5 to 100 KDa, (b) ethyl cellulose polymer having weight average molecular weight ranging from 100 to 200 KDa, (c) ethyl cellulose polymer having weight average molecular weight ranging from 200 to 300 KDa, and (d) ethyl cellulose polymer having weight average molecular weight ranging from 300 to 400 KDa; (ii) a blend of hydroxypropyl cellulose and at least one synthetic polymer; (iii) a blend of ethyl cellulose and at least one synthetic polymer; or (iv) a blend of ethyl cellulose; hydroxypropyl cellulose; and at least one synthetic polymer.

Each polymer ethyl cellulose, hydroxypropyl cellulose or synthetic polymer is present in an amount of 0.1 to 3.5 wt. % of the total hair spray composition.

Further, ethyl cellulose to hydroxypropyl cellulose blend is present in weight ratio of 0.1 to 1.0:0.1 to 1.0, or from 0.25 to 1:0.25 to 1.0. Hydroxypropyl cellulose to synthetic polymer is present in weight ratio of 0.1 to 1.0:0.25 to 1.0, 0.1 to 1.0. Ethyl cellulose to synthetic polymer is present in weight ratio of 0.25 to 1.0:0.25 to 1.0. Ethyl cellulose to hydroxypropyl cellulose to synthetic polymer is present in weight ratio of 0.25 to 1.0:0.25 to 1:0.25 to 1.0.

In another embodiment, the hydroxypropyl cellulose has a weight average molecular weight of from 10 to 400 KDa, or from 25 to 300 KDa, or from 50 to 200 KDa.

In another embodiment, the synthetic polymer has a weight average molecular weight of from 10 to 400 KDa, or from 25 to 300 KDa, or from 30 to 200 KDa.

In another embodiment, the composition has an average particle size distribution (D50) of at least 30 to 100 μm or of at least 40 to 95 μm.

In another embodiment, the present hair spray composition is an anhydrous composition or a hydroalcoholic composition.

In another embodiment, the hair spray composition on application to human hair provides High Humidity Curl Retention (HHCR) greater than 40% or greater than 70% or greater than 95% for at least 24 hours at 80 F (Fahrenheit), 90RH (Relative Humidity).

In another embodiment, the hair spray composition comprises a hair care additive selected from the group consisting of antistatic agents, amino acids, antioxidants, anticorrosion agents, bleaches, bodying agents, collagen, compressed gases, combing aids, conditioning agents, care agents, colorants, chelating agents, cleansing agents, clarifying agents, dyes, detackifying agents, defoamers, dispersible silicone-containing polymers, emollients, emulsifiers, fragrances, flexibility enhancers, film-forming polymers, fixative polymer, graft polymers, gel formers, glossifiers, gelling agents, humectants, hair polymers, hair and skin conditioners, lipids, lubricants, light protection agents, moisturizers, neutralizing agents, opacifiers, pigments, protein hydrolyzate, plasticizers, penetrants, perfumes, proteins, pearling agents, propellants, preservatives, refatting agents, shine enhancers, split modifiers, solubilizers, softeners, suspension agents, suspended materials, sequestering agents, stabilizers, surfactants, styling polymers, texturizing agents, tinting agents, tanning agents, thickeners, UV protectants, wetting agents, water-proofing agents, water-soluble silicone-containing polymers, and blends thereof.

Emollients deliver a lubricious and rich touch with a long-lasting afterfeel. Imparts cushion, improved playtime and rich skin feel. Emollients can include conventional lipids (e.g. esters such as fats, oils, waxes and the like), polar lipids (e.g. lipids that have been hydrophilically modified to render them more water soluble), silicones, and hydrocarbons. Particularly preferred emollients include distearyl adipate, Ceraphyl™ 791 ester (Isocetyl Stearoyl Stearate) by Ashland Specialty Chemical.

Film forming polymer are employed for aerosol and non-aerosol hair spray composition to provide strong, durable hold, stiff feel with good flexibility, superior high humidity curl retention at low solids level, excellent shine, high propellant tolerance without requirement of neutralization. Non-limiting examples of film forming polymers employed in the present application include vinyl caprolactam/vinyl pyrrolidone/dimethylaminoethyl methacrylate copolymer.

Non-limiting examples of cationic conditioning polymers used in the present application are selected from the group consisting of Chitosan, cocodimonium Hydroxypropyl Hydrolyzed Collagen, Cocodimonium Hydroxypropyl Hydrolyzed Hair Keratin, cocodimonium Hydroxypropyl Hydrolyzed Keratin, Polyquaternium-4, Polyquaternium-10, Cationic hydroxyethylcellulose, polyquaternium-6, polyquaternium-7, polyquaternium-39, polyquaternium-4, polyquaternium-10, and polyquaternium-44.

Suitable silicone conditioning agents that may be used in the present application include Water Soluble Silicones, Dimethicone Copolyol, Lauryl Methicone Copolyol, Hydrolyzed wheat protein (Hydroxypropyl Polysiloxane), Any Silicone with PEG as a prefix such as PEG/PPG-18/18 Dimethicone, Oil soluble Silicones, Trimethylsilyl amodimethicone, Dimethicone, Phenyl Trimethicone, Cetearyl Methicone, Dimethiconol, Amodimethicone, Stearyl Dimethicone, Cyclomethicone, Cetyl Dimethicone, cyclopentasiloxane, behenoxy Dimethicone and Stearoxy Dimethicone.

Hair fixative and styling polymers offer good sprayability, excellent high humidity curl retention and low-tack high propellant compatibility to the spray composition. Non-limiting examples of styling polymers employed in the present application include terpolymer of vinylcaprolactam, vinylpyrrolidone and dimethylaminopropyl methacrylamide. These styling or fixative polymers can be formulated at all volatile organic content (VOC) levels. It offers medium hold, has good sprayability and high propellant compatibility.

Non-limiting examples of vitamins and coenzymes that may be included in the present application is selected from the group comprising vitamin A, vitamin D, vitamin E, vitamin B1 (thiamine), B2 (riboflavin), B3 (niacin), B5 (pantothenic acid), B6 (pyridoxine), B8 (folic acid), B12 (Cobalamin), biotin, choline, lipoic acid, inositol, and the like. Vitamin B1 (thiamine), B2 (riboflavin), B3 (niacin), B5 (pantothenic acid), B6 (pyridoxine), B8 (folic acid), DL-Panthenol (ProVitamin B5), biotin, choline, lipoic acid, inositol, and myo-inositol. Vitamins can from 0.01 to 0.5 wt. % of the hair spray.

Minerals include calcium, sodium, potassium, magnesium, zinc, and aluminum slats of chlorides, hydrochlorides, carbonates, bicarbonates, nitrates, sulfates, monohydrogen phosphates, and dihydrogen phosphates. The total content of minerals is preferably 0.3 to 12% by mass, particularly preferably 0.5 to 8% by mass.

Pigments are used for color consistency and excellent dispersibility for the hair spray composition. Non-limiting examples of pigments employed in the present application include mineral pigments selected from organic pigments of various aromatic types including ado, indigoid, triphenylmethane, anthraquinone, and xanthine dyes which are designated as D&C and FD&C blues, browns, greens, oranges, reds, yellows, etc. Organic pigments generally consist of insoluble metallic salts of certified color additives, referred to as the Lakes. Inorganic pigments include iron oxides, ultramarine and chromium or chromium hydroxide colors, and mixtures thereof.

In another embodiment, the present application provides an aerosol hair spray composition comprising: (a) (i) 0.1 to 7.5 wt. % of a polymer blend comprising at least one nature-derived polymer selected from ethyl cellulose, hydroxypropyl cellulose and blends thereof; (ii) 0.01 to 50 wt. % of at least one hair care additive; and (iii) 0.1 to 99.9 wt. % of at least one solvent; and (b) at least one propellant.

Hair spray delivery systems will be blend of water and volatile organic compounds such as solvents or propellants. The total amount of volatile organic content will be limited by environmental regulations, between 80% to as less than 50% based on weight of the composition. Preferably, solvent will be a low boiling alcohol, acetal, ketone selected from the group consisting of methanol, ethanol, propanol, isopropanol, butanol, acetone, or dimethoxymethane.

A propellant is a liquefiable gas conventionally used for aerosol containers. Preferably the density of the propellant or mixture thereof is less than the hairspray concentrate so that pure propellant is not emitted from the container. Non-limiting examples of suitable propellants include dimethyl ether, propane, isopentane, n-butane, hydrofluorocarbon 152a, 1,1,1,3-tetrafluoro propene carbon dioxide, compressed air, nitrogen, nitrous oxide, and isobutane, used singly, or admixed. Dimethyl ether is preferred because of its water-solubility up to 35% by weight. The amount of the propellant gases is governed by normal factors well known in the aerosol art. For hairsprays the level of propellant is generally from about 3 to about 50%, preferably from about 5 to about 45%, optimally about 30% of the total composition.

The aerosol composition is actuated by a pump sprayer, a bag on valve system, a bag in can system, a sleeve in can system, or a piston system.

Attached to the container is a spraying device for spraying the hairstyling formulation onto a substrate. The device comprises the following components:

(i) valve—the spraying device comprises a valve. The valve comprises a valve body, said valve body comprising a stem and a spring means. The valve comprises a restricted tail piece (RTP) of diameter 0.1 mm to 2.5 mm, preferably 0.2 to 1.5 mm, most preferably 0.3 to 1.0 mm. The valve comprises a vapor phase tap (VPT) of diameter 0.1 mm to 1.5 mm, preferably 0.2 to 1.0 mm, most preferably 0.3 to 0.8 mm. When installed in the container, the valve is preferably seated in a valve cup (16). The valve cup (16) preferably comprises an outer gasket (17), which enables connection to the can by crimping onto a can bead; and an inner gasket (18). The inner gasket controls the release product when the actuator is pressed.
(ii) actuator—the spraying device comprises an actuator. The actuator typically sits over the stem. The actuator comprises a main spray channel and communicates with an insert.
(iii) insert—the insert comprises an exit orifice and from 1 to 8 sub-spray channels, preferably 4 to 6 sub-spray channels, most preferably 6 sub-spray channels; wherein said channels are tangentially disposed about the exit orifice. Suitable inserts are available from, for example, Coster. The insert controls the cone angle. A wide cone angle is preferred whilst maintaining the desirable particle size distribution.
(iv) channels—the sub-spray channels have a height of from 0.1 to 0.8, preferably from 0.3 to 0.7 mm and a length of from 0.1 to 0.5, preferably from 0.25 to 0.35 mm.
(v) exit orifice—the exit orifice is capable of being in liquid communication with the hairstyling formulation in the reservoir. The exit orifice has a diameter of from 0.01 to 2 mm, most preferably 0.5 mm; and an orifice thickness of from 0.1 to 1.5 mm, preferably from 0.3 to 1.0 mm; The orifice may be straight or conical in shape.

In another non-limiting embodiment, the present disclosure is directed to a method for styling hair, the method comprising applying to human hair, 0.11 to 4.5 wt. % of an aerosol composition comprising: (i) blend of (a) 0.1 to 5.0 wt. % of ethyl cellulose, and (b) 0.1 to 5.0 wt. % of at least one synthetic or nature-derived polymer selected from the group consisting of hydroxypropyl cellulose, vinyl caprolactam, polyvinylpyrrolidone (PVP), copolymers of vinyl pyrrolidone and methylmethacrylate, copolymers of vinyl pyrrolidone and vinyl acetate (VA), polyvinyl alcohol (PVA), hydroxypropyl guar gum, and mixtures; (iii) 0.1 to 50 wt. % of at least one hair care additive; and (iv) 0.1 to 99.9 wt. % of water, at least one solvent, at least one solvent system or combinations thereof; wherein the ratio of (a):(b) is 0.1 to 2.5:0.1 to 2.5, based on the total weight of the aerosol composition.

In another embodiment, the present application provides a non-aerosol hair spray composition comprising: (i) 0.1 to 7.5 wt. % of a polymer blend comprising at least one nature-derived polymer selected from ethyl cellulose, hydroxypropyl cellulose and blends thereof; (ii) 0.01 to 50 wt. % of at least one hair care additive; and (iii) 0.1 to 99.9 wt. % of at least one solvent.

Accordingly, the non-aerosol hair spray is a pump spray.

In another embodiment, the present application provides a method of styling hair, wherein the method comprises applying to human hair, 0.10 to 10.0 wt. % of a hair spray composition comprising: (i) 0.1 to 7.5 wt. % of a polymer blend comprising at least one nature-derived polymer selected from ethyl cellulose, hydroxypropyl cellulose and blends thereof; (ii) 0.1 to 99 wt. % of at least one solvent; and (iii) optionally 0.01 to 50 wt. % of at least one hair care additive.

To achieve a desired hair spray performance, the hair spray composition should have a viscosity of less than 50 cps at ambient temperature.

The present application provides hair spray compositions capable of being sprayed, exhibit rapid drying times when sprayed, and provide excellent holding power to hair, with minimal droop or tackiness under high humidity conditions. The holding power to hair of the compositions of this invention has been evaluated by measuring the Initial Curl Retention of hair tresses sprayed with the compositions and the Curl Retention after storage of the treated tresses at 90% relative humidity for up to 6 hours, herein after referred to “High Humidity Curl Retention”.

Preferably, the inventive hair spray compositions impart to human hair an Initial Curl Retention and the High Humidity Curl Retention of greater than about 15%, more preferably greater than about 40%, and most preferably greater than about 75%.

Further, certain aspects of the present application are illustrated in detail by way of the following examples. The examples are given herein for illustration of the application and are not intended to be limiting thereof.

### EXAMPLES

### Comparative Examples

### Only HPC/Only EC—Control Samples

Control samples of Hydroxypropyl Cellulose and Ethyl cellulose alone were prepared comparative studies.

TABLE 1
Only EC/HPC

| | Example 1 | Example 2 |
|---|---|---|
| | Amount | Amount |
| Ingredients | (wt. %) | (wt. %) |
| Ratio HPC:EC | 1:0 | 0:1 |
| Hydroxypropyl Cellulose | 2.25 | — |
| (MW: 10-100 KD) | | |
| Hydroxypropyl Cellulose | 0.25 | — |
| (MW: 110-400 KD) | | |
| Ethyl Cellulose | — | 2.0 |
| (MW: 110-400 KD) | | |

(MW: molecular weight)

### Hydroxypropyl Cellulose—Ethyl Cellulose Blends

Polymers blends were prepared, wherein the polymers ethyl cellulose and hydroxypropyl cellulose with different weight average molecular weights were combined in different weight ratios.

TABLE 2
Ethyl cellulose and Hydroxypropyl cellulose blends

| | Example 3 | Example 4 | Example 5 | Example 6 |
|---|---|---|---|---|
| | Amount | Amount | Amount | Amount |
| Ingredients | (wt. %) | (wt. %) | (wt. %) | (wt. %) |
| HPC/EC Ratio | 1.0:0.25 | 1.0:0.375 | 1.0:0.375 | 0.5:1.0 |
| Hydroxypropyl | 1.75 | 1.75 | 1.75 | 0.75 |
| Cellulose | | | | |
| (MW: 10-100 KD) | | | | |
| Hydroxypropyl | 0.25 | 0.25 | 0.25 | 0.25 |
| Cellulose | | | | |
| (MW: 110-400 KD) | | | | |
| Ethyl Cellulose | 0.5 | 0.75 | — | 2.0 |
| (MW: 10-100 KD) | | | | |
| Ethyl Cellulose | — | — | 0.75 | — |
| (MW: 110-400 KD) | | | | |

### Hydroxypropyl Cellulose—Ethyl Cellulose Blends

Blends of hydroxypropyl cellulose and ethyl cellulose were prepared, wherein Ethyl cellulose and hydroxypropyl cellulose were combined in different weight average molecular weight distribution.

TABLE 3
Hydroxypropyl Cellulose - Ethyl Cellulose Blends

| | Example 7 | Example 8 | Example 9 | Example 10 |
|---|---|---|---|---|
| | Amount | Amount | Amount | Amount |
| Ingredients | (wt. %) | (wt. %) | (wt. %) | (wt. %) |
| HPC/EC Ratio | 0.25:1.0 | 1.0:0.9 | 1.0:0.25 | 2.5:1 |
| Hydroxypropyl | 0.35 | 1.125 | 2.0 | 5.0 |
| Cellulose | | | | |
| (MW: 10-100 KD) | | | | |
| Hydroxypropyl | 0.15 | 0.125 | — | — |
| Cellulose | | | | |
| (MW: 110-400 KD) | | | | |
| Ethyl Cellulose | 2.0 | 1.0 | 0.5 | 2.0 |
| (MW: 10-100 KD) | | | | |
| Ethyl Cellulose | — | 0.375 | — | — |
| (MW: 110-400 KD) | | | | |
| Ethanol | — | — | 57.25 | 57.50 |
| Dimethyl ether | — | — | 40 | 40 |

(MW: molecular weight)

### Examples 11 to 18: Aerosol Ethyl Cellulose Blends

Aerosol system using ethyl cellulose blends with different weight average molecular weight distributions were prepared.

TABLE 4
Ethyl Cellulose Blends

| | Ex. 11 | Ex. 12 | Ex. 13 | Ex. 14 | Ex. 15 | Ex. 16 | Ex. 17 | Ex. 18 |
|---|---|---|---|---|---|---|---|---|
| | Amount | | | | | | | |
| Ingredients | wt. % | wt. % | wt. % | wt. % | wt. % | wt. % | wt. % | wt. % |
| Ethanol (denatured) | 57.50 | 50.00 | 57.50 | 57.50 | 50.00 | 52.50 | 97.50 | 57.43 |
| water | — | — | — | — | — | 5.00 | — | — |
| Ethyl Cellulose | 2.00 | 1.50 | 2.00 | 1.00 | 1.00 | 0.50 | 0.50 | 0.50 |
| (MW: 150-250k) | | | | | | | | |
| Ethyl Cellulose | — | — | — | 0.50 | 0.50 | 1.50 | 1.50 | 1.50 |
| (MW: 50-150k) | | | | | | | | |
| Ethyl Cellulose | 0.25 | 0.50 | 0.25 | 0.50 | 0.50 | 0.25 | 0.25 | 0.25 |
| (MW: 15-50k) | | | | | | | | |
| Ethyl Cellulose | 0.25 | 0.50 | 0.25 | 0.50 | 0.50 | 0.25 | 0.25 | 0.25 |
| (MW: 5-15k) | | | | | | | | |
| Lauryl Pyrrolidone | — | — | — | — | — | — | — | 0.08 |
| Citric Acid | — | — | — | — | — | — | — | — |
| lactic acid | — | — | — | — | — | — | — | — |
| polylysine | — | — | — | — | — | — | — | — |
| esters of citric acid | — | — | — | — | — | — | — | — |
| Sodium hydroxide(50% soln) | — | — | — | — | — | — | — | — |
| ethoxylated silicone | — | — | — | — | — | — | — | — |
| caprylyl glycol | — | — | — | — | — | — | — | — |
| hydrofluoro olefin | — | — | — | — | 47.50 | — | — | — |
| Hydrocarbon | — | — | — | 40.00 | — | — | — | — |
| hydrofluorocarbon 152a | — | 47.5 | — | — | — | — | — | — |
| Dimethyl Ether | 40.00 | — | 40.00 | — | — | 40.00 | — | 40.00 |
| Total | 100.00 | 100.00 | 100.00 | 100.00 | 100.00 | 100.00 | 100.00 | 100.00 |

(MW: molecular weight)

### Examples 19 to 26—Non-Aerosol Ethyl Cellulose Blends

Ethyl cellulose blends were prepared with ethyl cellulose having different weight average molecular weight distributions.

TABLE 5
Ethyl Cellulose blends

| | Ex. 19 | Ex. 20 | Ex. 21 | Ex. 22 | Ex. 23 | Ex. 24 | Ex. 25 | Ex. 26 |
|---|---|---|---|---|---|---|---|---|
| | Amount | | | | | | | |
| Ingredients | wt. % | wt. % | wt. % | wt. % | wt. % | wt. % | wt. % | wt. % |
| Ethanol (denatured) | 57.00 | 57.00 | 57.00 | 51.50 | 56.50 | 55.93 | 95.73 | 90.73 |
| water | — | — | — | 5.00 | — | — | — | 5.00 |
| Ethyl Cellulose | 0.50 | 0.50 | 0.50 | 0.50 | 0.50 | 0.50 | 0.50 | 0.50 |
| (MW: 150-250k) | | | | | | | | |
| Ethyl Cellulose | 1.50 | 1.50 | 1.50 | 1.50 | 1.50 | 1.50 | 1.50 | 1.50 |
| (MW: 50-150k) | | | | | | | | |
| Ethyl Cellulose | 0.25 | 0.25 | 0.25 | 0.25 | 0.25 | 0.25 | 0.25 | 0.25 |
| (MW: 15-50k) | | | | | | | | |
| Ethyl Cellulose | 0.25 | 0.25 | 0.25 | 0.25 | 0.25 | 0.25 | 0.25 | 0.25 |
| (MW: 5-15k) | | | | | | | | |
| Lauryl Pyrrolidone | — | — | — | — | — | 0.08 | 0.08 | 0.08 |
| Citric Acid | 0.50 | — | — | — | — | — | — | — |
| lactic acid | — | — | 0.50 | — | — | — | — | — |
| polylysine | — | — | — | 1.00 | — | — | — | — |
| esters of citric acid | — | — | — | — | 1.00 | 1.00 | 1.00 | 1.00 |
| Sodium hydroxide(50% soln) | — | 0.50 | — | — | — | — | — | — |
| ethoxylated silicone | — | — | — | — | — | 0.50 | 0.50 | 0.50 |
| caprylyl glycol | — | — | — | — | — | — | 0.20 | 0.20 |
| hydrofluoro olefin | — | — | — | — | — | — | — | — |
| Hydrocarbon | — | — | — | — | — | — | — | — |
| Dimethyl Ether | 40.00 | 40.00 | 40.00 | 40.00 | 40.00 | 40.00 | — | — |
| Total | 100.00 | 100.00 | 100.00 | 100.00 | 100.00 | 100.00 | 100.00 | 100.00 |

(MW: molecular weight)

### Examples 27 to 28—Non-Aerosol Pump System

A non-aerosol pump systems was prepared using Ethyl cellulose and hydroxypropyl cellulose blends.

TABLE 6
Non-Aerosol pump system

| | Ex. 27 | Ex. 28 |
|---|---|---|
| Ingredients | Amount (Wt. %) | Amount (wt. %) |
| Ethanol | 97.375 | 90.0 |
| Hydroxypropyl Cellulose | 1.125 | 1.125 |
| (MW: 10-100 KD) | | |
| Hydroxypropyl Cellulose | 0.125 | 0.125 |
| (MW: 110-400 KD) | | |
| Ethyl Cellulose | 1.375 | 1.375 |
| (MW: 10-100 KD) | | |
| Water | — | 7.375 |
| Total | 100 | 100 |

(MW: molecular weight)

### Examples 29 to 33: Aerosol Hair Spray Composition with Synthetic Polymers

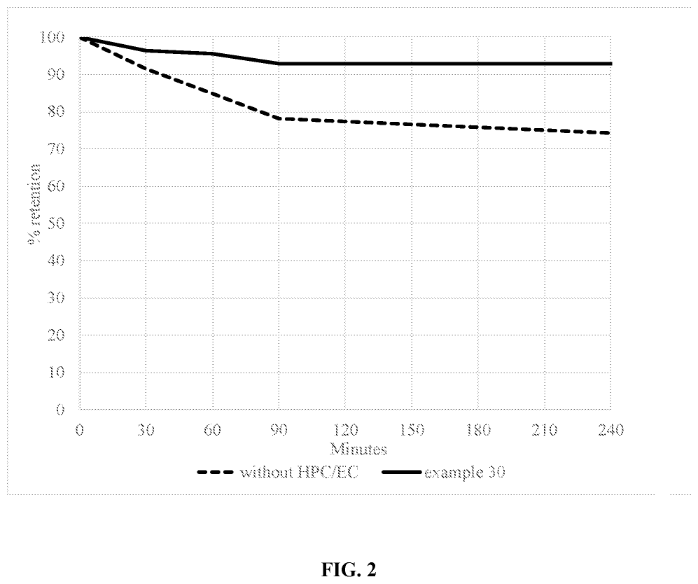

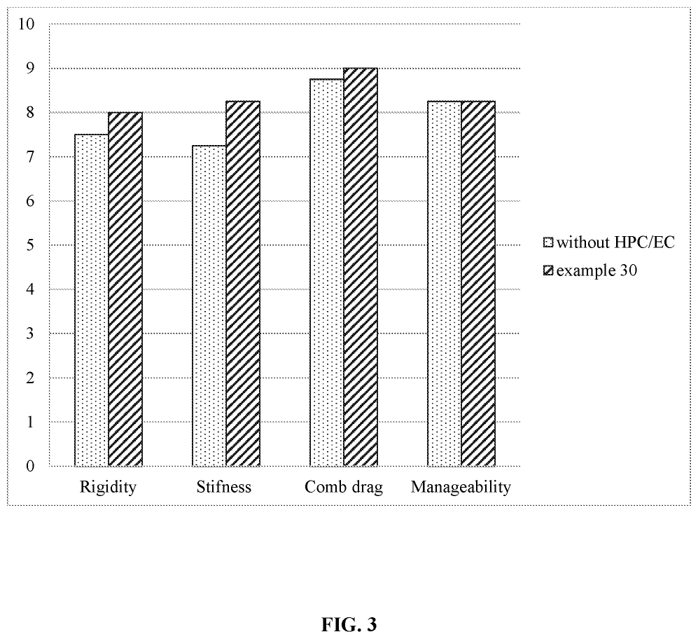

Hair sprays with Ethyl cellulose (EC), hydroxypropyl cellulose (HPC) and synthetic polymers in different weight average molecular weight distributions were prepared. Results for Aerosol hair spray of example 30 is shown in FIGS. 2 & 3 accordingly. FIG. 2 represents humidity resistance of HPC/EC blends with a synthetic polymer at 80 F (Fahrenheit) and 90% RH (Relative Humidity) showing higher retention values. FIG. 3 represents on hair performance of HPC/EC blend with a synthetic polymer, wherein hair characteristics such as rigidity, stiffness, comb drag, and manageability show increased values.

TABLE 7
Aerosol with EC, HPC and Synthetic polymers

| | Ex. 29 | Ex. 30 | Ex. 31 | Ex. 32 | Ex. 33 |
|---|---|---|---|---|---|
| | Amount | Amount | Amount | Amount | Amount |
| Ingredients | (wt. %) | (wt. %) | (wt. %) | (wt. %) | (wt. %) |
| Ethanol | 49.9 | 51.0 | 48.20 | 46.90 | 64.45 |
| Water | 5.0 | — | — | — | 4.00 |
| Aminomethyl propanol | 0.91 | 0.28 | — | — | — |
| Hydroxypropyl Cellulose | 0.10 | — | 0.15 | — | 0.15 |
| (MW: 110-400 KD) | | | | | |
| Hydroxypropyl Cellulose | 0.35 | 0.25 | 0.35 | 0.35 | 0.30 |
| (MW: 10-100KD) | | | | | |
| Ethyl Cellulose (MW: | — | 0.25 | — | 0.10 | 0.10 |
| 110-400KD) | | | | | |
| Ethyl Cellulose (MW: | 0.05 | — | 0.10 | — | — |
| 10-100KD) | | | | | |
| Octylacrylamide/ | 4.50 | — | — | — | — |
| Acrylates/Butyl | | | | | |
| aminoethyl Meth- | | | | | |
| acrylate Copolymer | | | | | |
| Vinyl Caprolactam/PVP/ | — | — | 10.80 | 8.10 | — |
| Dimethyl aminoethyl | | | | | |
| Methacrylate Copolymer | | | | | |
| VP/Vinylcaprolactam/ | — | — | — | 5.00 | — |
| DMAPA Acrylates | | | | | |
| Copolymer | | | | | |
| Ethyl ester of PVM/ | — | 8.0 | — | — | — |
| MA Copolymer | | | | | |
| Polyvinyl pyrrolidone | — | — | 0.10 | — | — |
| VP/VA copolymer | — | — | — | — | 6.0 |
| Hydrofluoro carbon | — | 40.22 | 40.30 | — | — |
| Hydrocarbon (LPG) | — | — | — | 39.55 | 25 |
| Dimethyl ether | 40 | — | — | — | — |
| Total | 100 | 100 | 100 | 100 | 100 |
| Solids | 5.00 | 4.5 | 4.7 | 5.45 | 3.55 |
| % VOC | 89.09 | 55.0 | 55.0 | 55.00 | 67.45 |

(MW: molecular weight)

### Examples 34 to 38: Aerosol Hair Spray Composition with Synthetic Polymers

Aerosol Hair sprays with Ethyl Cellulose (EC), Hydroxypropyl cellulose (HPC) and synthetic polymers in different concentrations were prepared.

TABLE 8
Aerosol with EC, HPC and Synthetic polymers

| | Ex. 34 | Ex. 35 | Ex. 36 | Ex. 37 | Ex. 38 |
|---|---|---|---|---|---|
| | Amount | Amount | Amount | Amount | Amount |
| Ingredients | (wt. %) | (wt. %) | (wt. %) | (wt. %) | (wt. %) |
| Ethanol | 51.50 | 55.0 | 66.62 | 52.32 | 95.40 |
| Water | — | — | 4.0 | — | — |
| Aminomethyl propanol | — | 0.97 | 0.38 | 0.38 | — |
| Hydroxypropyl Cellulose | 0.15 | — | — | — | 0.15 |
| (MW: 110-400KD) | | | | | |
| Hydroxypropyl Cellulose | 0.30 | 0.35 | 0.30 | 0.25 | 0.70 |
| (MW: 10-100KD) | | | | | |
| Ethyl Cellulose (MW: | — | — | — | — | — |
| 110-400KD) | | | | | |
| Ethyl Cellulose (MW: | 0.10 | 0.15 | 0.20 | 0.25 | 0.05 |
| 10-100KD) | | | | | |
| Acrylates Copolymer | — | 4.00 | — | — | — |
| Polyurethane-14 (and) | — | — | — | 10.70 | — |
| AMP-Acrylates | | | | | |
| Copolymer | | | | | |
| VA/Crotonates/Vinyl | — | — | 3.50 | — | — |
| Neodecanoate | | | | | |
| Copolymer | | | | | |
| terpolymer of | — | — | — | — | 2.50 |
| vinylcaprolactam, | | | | | |
| vinylpyrrolidone and | | | | | |
| dimethylaminopropyl | | | | | |
| methacrylamide | | | | | |
| Vinyl Caprolactam/PVP/ | — | — | — | — | — |
| Dimethyl aminoethyl | | | | | |
| Methacrylate Copolymer | | | | | |
| Ethyl ester of PVM/MA | — | — | — | — | 0.20 |
| Copolymer | | | | | |
| Polyvinyl pyrrolidone | — | — | — | — | — |
| VP/VA copolymer | 7.00 | — | — | — | — |
| Hydrofluoro carbon | | 39.53 | 25.0 | 36.10 | — |
| Dimethyl ether | — | — | — | — | — |
| 1,1,1,3-tetrafluoro | 40.95 | — | — | — | — |
| propene | | | | | |
| Nitrogen | — | — | — | — | 1.0 |
| Total | 100 | 100 | 100 | 100 | 100 |
| Solids | 4.05 | 4.50 | 4.0 | 3.50 | 2.0 |
| % VOC | 55.00 | 55.00 | 66.62 | 55.00 | 97.0 |

(MW: molecular weight)

### Example 39 to 44: Hair Spray Composition

Aerosol Hair sprays with EC, HPC in different concentrations were prepared.

TABLE 9
Hair Spray

| | Ex: 39 | Ex: 40 | Ex: 41 | Ex: 42 | Ex: 43 | Ex: 44 |
|---|---|---|---|---|---|---|
| Ingredients | wt. % | wt. % | wt. % | wt. % | wt. % | wt. % |
| Ethanol | 56.375 | 57.200 | 57.375 | 57.125 | 57.125 | 51.725 |
| Water | — | — | — | — | — | 5.000 |
| Ethyl cellulose | 1.000 | 2.000 | 1.000 | 1.000 | 1.000 | 1.375 |
| (MW: 10-100KD); DS - 0.3 | | | | | | |
| Ethyl cellulose | 0.375 | — | 0.375 | 0.375 | 0.375 | — |
| (MW: 110-400KD); DS - 0.3 | | | | | | |
| Hydroxypropyl cellulose | 1.125 | 0.800 | 1.125 | 1.125 | 1.125 | 1.125 |
| (MW: 110-400KD); DS - 0.3 | | | | | | |
| Hydroxypropyl cellulose | 1.125 | — | 0.125 | 0.125 | 0.125 | 0.125 |
| (MW: 10-100KD); DS - 0.3 | | | | | | |
| DL Panthenol | — | — | — | 0.250 | — | — |
| Butylene Glycol | — | — | — | — | 0.250 | 0.250 |
| Diisopropyl adipate | — | — | — | — | — | 0.150 |
| PEG/PPG-18/18 | — | — | — | — | — | 0.250 |
| Dimethicone | | | | | | |
| Dimethyl ether | 40.000 | 40.000 | 40.000 | 40.000 | 40.000 | 40.000 |
| Total | 100.000 | 100.000 | 100.000 | 100.000 | 100.000 | 100.000 |

(MW: molecular weight)

### Example 45 to 48: Root Touch-Up Hair Spray

A root touch-up hair sprays were prepared using the following ingredients.

TABLE 10
Root Touch up Hair Spray

| | Ex: 45 | Ex: 46 | Ex: 47 | Ex: 48 |
|---|---|---|---|---|
| Ingredients | wt. % | wt. % | wt. % | wt. % |
| Ethanol | 7.19 | 7.19 | 9.00 | 9.00 |
| Ethyl Trisiloxane | — | — | — | — |
| Isocetyl Stearoyl Stearate | 0.50 | 0.50 | — | — |
| Vinyl Caprolactam/PVP/Dimethyl | 0.68 | 0.68 | — | — |
| aminoethyl Methacrylate | | | | |
| Copolymer | | | | |
| PVP/Vinylcaprolactam/DMAPA | 0.63 | 0.63 | — | — |
| Acrylates Copolymer | | | | |
| Iron Oxide LC989 (Blk) | 0.50 | — | 0.50 | — |
| Iron Oxide LC989 AS (Blk) | — | 0.50 | — | 0.50 |
| Ethyl cellulose (MW: 10-100k) | 0.50 | 0.50 | 0.50 | 0.50 |
| Isobutane | 46.50 | 46.50 | 46.50 | 46.50 |
| Hydrofluorocarbon 152a | 43.50 | 43.50 | 43.50 | 43.50 |
| Total | 100.00 | 100.00 | 100.00 | 100.00 |

On hair performance as determined by shine, stiffness, hold and resultant humidity resistance based on the individual components and the ability of blends to balance attributes. (Table 1, examples 1 and 2; Table 2 example 3, 4, 5 and 6; Table 3 example 7 and 8).

### Examples 48 to 55—Non-Aerosol Blends with Synthetic Polymers

Non aerosol blends with synthetic polymers were prepared.

TABLE 11
Non Aerosol Blends

| | Ex. 49 | Ex. 50 | Ex. 51 | Ex. 52 | Ex. 53 | Ex. 54 | Ex. 55 |
|---|---|---|---|---|---|---|---|
| Ingredients | wt. % | wt. % | wt. % | wt. % | wt. % | wt. % | wt. % |
| Ethanol | 91.27 | 91.27 | 91.27 | 91.62 | 86.27 | 46.40 | 84.43 |
| Water | — | — | — | — | 5.00 | 43.00 | 10.00 |
| Aminomethyl propanol | 0.23 | 0.23 | 0.23 | 0.23 | 0.23 | — | 0.82 |
| Hydroxypropyl cellulose MW: 200-300K | — | 0.15 | 0.10 | 0.10 | 0.10 | 0.10 | 0.10 |
| Hydroxypropyl cellulose MW: 50-100K | — | 0.35 | 0.25 | — | 0.25 | 0.25 | 0.15 |
| Ethyl cellulose MW: 150-250K | 0.50 | — | 0.15 | 0.75 | 0.15 | — | — |
| Ethyl ester of PVM/MA Copolymer (50% | 8.00 | 8.00 | 8.00 | 7.30 | 8.00 | — | — |
| in ethanol) | | | | | | | |
| Isobutylene/Ethylmaleimide/Hydroxyethyl | — | — | — | — | — | 10.25 | — |
| Maleimide Copolymer (40% in | | | | | | | |
| ethanol/water) | | | | | | | |
| Octylacrylamide/Acrylates/Butyl | — | — | — | — | — | — | 4.50 |
| aminoethyl Methacrylate Copolymer 100% | | | | | | | |
| Total | 100.00 | 100.00 | 100.00 | 100.00 | 100.00 | 100.00 | 100.00 |

(MW: molecular weight)

While the compositions and methods of the disclosed and/or claimed inventive concept(s) have been described in terms of particular aspects, it will be apparent to those of ordinary skill in the art that variations may be applied to the compositions and/or methods and in the steps or in the sequence of steps of the method described herein without departing from the concept, spirit and scope of the disclosed and/or claimed inventive concept(s). All such similar substitutes and modifications apparent to those skilled in the art are deemed to be within the spirit, scope, and concept of the disclosed! and/or claimed inventive concept(s).

What is claimed is:

1. A hair spray composition comprising:
(i) 0.1 to 7.5 wt. % of a polymer blend comprising at least one nature-derived polymer selected from ethyl cellulose, hydroxypropyl cellulose and blends thereof;
(ii) 0.1 to 99 wt. % of at least one solvent; and
(iii) optionally 0.01 to 50 wt. % of at least one hair care additive.

2. The hair spray composition according to claim 1, wherein the hair spray composition further optionally comprises 0.1 to 5.0 wt. % of at least one synthetic polymer selected from the group consisting of vinyl pyrrolidone/vinyl caprolactam/dimethylaminoethyl methacrylate copolymer, vinyl acetate/mono n-butyl maleate/isobornyl acrylate copolymer, N-tert-octylacrylamide, methylmethacrylate/acrylic acid/hydroxypropyl methacrylate/butyl aminoethyl methacrylate polymer, isobutylene/imidized maleic anhydride with ethylamine and ethanolamine, vinyl pyrrolidone/vinyl caprolactam/dimethylaminopropyl acrylamide copolymer, alkylated pyrrolidone (butyl), methyl vinyl ether/half ester of maleic anhydride (ethyl or butyl), vinyl pyrrolidone (PVP)/vinyl acetate/vinyl pyrrolidone copolymer, hydroxypropyl cellulose, hydroxethyl cellulose, octylacrylamide/acrylates/butyl aminoethyl methacrylate copolymer, acrylates/octylacrylamide copolymer, acrylates copolymer, Polyurethane-14 and AMP-acrylates copolymer, acrylates/hydroxypropyl epoxidized soybean oil copolymer, vinyl acetate/crotonates/vinyl neodecanoate copolymer, acrylates/octylacrylamide copolymer, t-butyl acrylate/ethyl acrylate/methacrylic acid, t-butyl acrylate/methacrylic acid/Dimethicone Copolyol, vinyl acetate/crotonic acid/vinyl neodecanoate, vinyl acetate/crotonic acid copolymer, copolymer of isophthalic acid, adipic acid, hexylene glycol, neopentyl glycol, dimethylol propanoic acid, and isophorone diisocyanate monomers, Polyacrylate-22, polyvinyl caprolactam, acrylates/t-butylacrylamide copolymer, acrylates/hydroxyesters acrylates copolymer, acrylates/C1-2 succinates/hydroxy acrylates copolymer, styrene/acrylates copolymer, pyrrolidone carboxylic acid salt of Chitosan, acrylates copolymer, methacryloyl ethyl betaine/acrylates copolymer, methacryloyl ethyl betaine/acrylates copolymer, and Polyester-5.

3. The hair spray composition according to claim 1, wherein the polymer blend is 0.1 to 5.0 wt. % of ethyl cellulose and hydroxypropyl cellulose.

4. The hair spray composition according to claim 1, wherein the polymer is
(i) a blend of ethyl cellulose polymers selected from the group consisting of
(a) ethyl cellulose polymer having a weight average molecular weight ranging from 5 to 100 KDa,
(b) ethyl cellulose polymer having a weight average molecular weight ranging from 100 to 200 KDa,
(c) ethyl cellulose polymer having a weight average molecular weight ranging from 200 to 300 KDa, and
(d) ethyl cellulose polymer having a weight average molecular weight ranging from 300 to 400 KDa;
(ii) a blend of hydroxypropyl cellulose and at least one synthetic polymer of claim 2;
(iii) a blend of ethyl cellulose and at least one synthetic polymer of claim 2; or
(iv) a blend of ethyl cellulose; hydroxypropyl cellulose; and at least one synthetic polymer of claim 2.

5. The hair spray composition according to claim 1, wherein the composition is an anhydrous composition or a hydroalcoholic composition.

6. The hair spray composition according to claim 1, wherein the ethyl cellulose is present in an amount of 0.1 to 3.5 wt. % of the total hair spray composition.

7. The hair spray composition according to claim 1, wherein the hydroxypropyl cellulose is present in an amount of 0.1 to 3.5 wt. % of the total hair spray composition.

8. The hair spray composition according to claim 1, wherein the synthetic polymer is present in an amount of 0.1 to 3.5 wt. % of the total hair spray composition.

9. The hair spray composition according to claim 1, wherein the weight ratio of ethyl cellulose to hydroxypropyl cellulose is 0.1 to 1:0.1 to 1.0.

10. The hair spray composition according to claim 4, wherein the weight ratio of:
(i) hydroxypropyl cellulose to synthetic polymer is 0.1 to 1.0:0.25 to 1.0, 0.1 to 1.0;
(ii) ethyl cellulose to synthetic polymer is 0.25 to 1.0:0.25 to 1.0; or
(iii) ethyl cellulose to hydroxypropyl cellulose to synthetic polymer is 0.25 to 1.0:0.25 to 1:0.25 to 1.0.

11. The hair spray composition according to claim 1, wherein the hydroxypropyl cellulose has a weight average molecular weight of from 10 to 400 KDa, or from 25 to 300 KDa, or from 50 to 200 KDa.

12. The hair spray composition according to claim 2, wherein the synthetic polymer has a weight average molecular weight of from 10 to 400 KDa, or from 25 to 300 KDa, or from 30 to 200 KDa.

13. The hair spray composition according to claim 1, wherein the composition has an average particle size distribution (D50) of at least 30 to 100 or of at least 40 to 95.

14. The hair spray composition according to claim 1, wherein the composition on application to human hair provides High Humidity Curl Retention (HHCR) greater than 40% or greater than 70% or greater than 95% for at least 24 hours at 80F, 90RH.

15. The hair spray composition according to claim 1, wherein the hair care additive is selected from the group consisting of antistatic agents, amino acids, antioxidants, anticorrosion agents, bleaches, bodying agents, collagen, compressed gases, combing aids, conditioning agents, care agents, colorants, chelating agents, cleansing agents, clarifying agents, dyes, detackifying agents, defoamers, dispersible silicone-containing polymers, emollients, emulsifiers, fragrances, flexibility enhancers, film-forming polymers, fixative polymer, graft polymers, gel formers, glossifiers, gelling agents, humectants, hair polymers, hair and skin conditioners, lipids, lubricants, light protection agents, moisturizers, neutralizing agents, opacifiers, pigments, protein hydrolyzate, plasticizers, penetrants, perfumes, proteins, pearling agents, propellants, preservatives, refatting agents, shine enhancers, split modifiers, solubilizers, solvents, softeners, suspension agents, suspended materials, sequestering agents, stabilizers, surfactants, styling polymers, texturizing agents, tinting agents, tanning agents, thickeners, UV protectants, wetting agents, water-proofing agents, water-soluble silicone-containing polymers, and blends thereof.

16. The hair spray composition according to claim 16, wherein the propellant is selected from the group consisting of dimethyl ether, isopentane, n-butane, isobutane, propane, hydrofluorocarbon 152a, 1,1,1,3-tetrafluoro propene, carbon dioxide, compressed air, nitrogen, nitrous oxide or mixtures thereof.

17. The hair spray composition according to claim 1, wherein the solvent comprises esters, polyols.

18. The hair spray composition according to claim 17, wherein the solvent is selected from the group consisting of ethanol, ethyl acetate, isododecane, isohexadecane, isopropyl alcohol (IPA), methylal, methyl acetate, water, and mixtures thereof.

19. The hair spray composition according to claim 17, wherein the solvent comprises ethanol.

20. The hair spray composition according to claim 1, wherein the composition is an aerosol or a non-aerosol hair spray.

21. A method for styling hair, the method comprising applying to human hair, 0.10 to 10.0 wt. % of a hair spray composition of claim 1.

22. The method according to claim 21, wherein the aerosol composition is actuated by a pump sprayer, a bag on valve system, a bag in can system, a sleeve in can system, or a piston system.

23. An aerosol hair spray composition comprising:
(a) (i) 0.1 to 7.5 wt. % of a polymer blend comprising at least one nature-derived polymer selected from ethyl cellulose, hydroxypropyl cellulose and blends thereof;
(ii) 0.01 to 50 wt. % of at least one hair care additive; and
(iii) 0.1 to 99.9 wt. % of at least one solvent; and
(b) at least one propellant.

24. The aerosol hair spray composition according to claim 23, wherein the hair spray composition optionally further comprises 0.1 to 7.5 wt. % of at least one synthetic polymer selected from the group consisting of vinyl pyrrolidone/vinyl caprolactam/dimethylaminoethyl methacrylate copolymer, vinyl acetate/mono n-butyl maleate/isobornyl acrylate copolymer, N-tert-octylacrylamide, methylmethacrylate/acrylic acid/hydroxypropyl methacrylate/butyl aminoethyl methacrylate polymer, isobutylene/imidized maleic anhydride with ethylamine and ethanolamine, vinyl pyrrolidone/vinyl caprolactam/dimethylaminopropyl acrylamide copolymer, alkylated pyrrolidone (butyl), methyl vinyl ether/half ester of maleic anhydride (ethyl or butyl), vinyl pyrrolidone (PVP)/vinyl acetate/vinyl pyrrolidone copolymer, hydroxypropyl cellulose, hydroxethyl cellulose, octylacrylamide/acrylates/butyl aminoethyl methacrylate copolymer, acrylates/octylacrylamide copolymer, acrylates copolymer, Polyurethane-14 and AMP-acrylates copolymer, acrylates/hydroxypropyl epoxidized soybean oil copolymer, vinyl acetate/crotonates/vinyl neodecanoate copolymer, acrylates/octylacrylamide copolymer, t-butyl acrylate/ethyl acrylate/methacrylic acid, t-butyl acrylate/methacrylic acid/Dimethicone Copolyol, vinyl acetate/crotonic acid/vinyl neodecanoate, vinyl acetate/crotonic acid copolymer, copolymer of isophthalic acid, adipic acid, hexylene glycol, neopentyl glycol, dimethylol propanoic acid, and isophorone diisocyanate monomers, Polyacrylate-22, polyvinyl caprolactam, acrylates/t-butylacrylamide copolymer, acrylates/hydroxyesters acrylates copolymer, acrylates/C1-2 succinates/hydroxy acrylates copolymer, styrene/acrylates copolymer, pyrrolidone carboxylic acid salt of chitosan, acrylates copolymer, methacryloyl ethyl betaine/acrylates copolymer, methacryloyl ethyl betaine/acrylates copolymer, and Polyester-5.

25. A non-aerosol hair spray composition comprising:
(a) (i) 0.1 to 7.5 wt. % of a polymer blend comprising at least one nature-derived polymer selected from ethyl cellulose, hydroxypropyl cellulose and blends thereof;
(ii) 0.01 to 50 wt. % of at least one hair care additive; and
(iii) 0.1 to 99.9 wt. % of at least one solvent.

26. The non-aerosol hair spray composition according to claim 25, wherein the hair spray composition optionally further comprises 0.1 to 7.5 wt. % of at least one synthetic polymer selected from the group consisting of vinyl pyrrolidone/vinyl caprolactam/dimethylaminoethyl methacrylate copolymer, vinyl acetate/mono n-butyl maleate/isobornyl acrylate copolymer, N-tert-octylacrylamide, methylmethacrylate/acrylic acid/hydroxypropyl methacrylate/butyl aminoethyl methacrylate polymer, isobutylene/imidized maleic anhydride with ethylamine and ethanolamine, vinyl pyrrolidone/vinyl caprolactam/dimethylaminopropyl acrylamide copolymer, alkylated pyrrolidone (butyl), methyl vinyl ether/half ester of maleic anhydride (ethyl or butyl), vinyl pyrrolidone (PVP)/vinyl acetate/vinyl pyrrolidone copolymer, hydroxypropyl cellulose, hydroxethyl cellulose, octylacrylamide/acrylates/butyl aminoethyl methacrylate copolymer, acrylates/octylacrylamide copolymer, acrylates copolymer, Polyurethane-14 and AMP-acrylates copolymer, acrylates/hydroxypropyl epoxidized soybean oil copolymer, vinyl acetate/crotonates/vinyl neodecanoate copolymer, acrylates/octylacrylamide copolymer, t-butyl acrylate/ethyl acrylate/methacrylic acid, t-butyl acrylate/methacrylic acid/Dimethicone Copolyol, vinyl acetate/crotonic acid/vinyl neodecanoate, vinyl acetate/crotonic acid copolymer, copolymer of isophthalic acid, adipic acid, hexylene glycol, neopentyl glycol, dimethylol propanoic acid, and isophorone diisocyanate monomers, Polyacrylate-22, polyvinyl caprolactam, acrylates/t-butylacrylamide copolymer, acrylates/hydroxyesters acrylates copolymer, acrylates/C1-2 succinates/hydroxy acrylates copolymer, styrene/acrylates copolymer, pyrrolidone carboxylic acid salt of chitosan, acrylates copolymer, methacryloyl ethyl betaine/acrylates copolymer, methacryloyl ethyl betaine/acrylates copolymer, and Polyester-5.

27. The non-aerosol hair spray composition according to claim 25, wherein the non-aerosol hair spray is a pump spray.
